(19) United States
(12) Patent Application Publication
(10) Pub. No.: US 2024/0268735 A1
(43) Pub. Date: Aug. 15, 2024

(54) DEVICE, METHOD AND SYSTEM FOR DETECTING OVARIAN CANCER

(71) Applicants: Emanuele Raggi, Providence, RI (US); Alessandro Maria Sabatini, Spoleto (IT)

(72) Inventors: Emanuele Raggi, Providence, RI (US); Alessandro Maria Sabatini, Spoleto (IT)

(21) Appl. No.: 18/437,503

(22) Filed: Feb. 9, 2024

Related U.S. Application Data
(60) Provisional application No. 63/484,068, filed on Feb. 9, 2023.

(51) Int. Cl. A61B 5/20; G01N 27/414; G16H 40/67

(52) U.S. Cl. CPC A61B 5/207; G01N 27/4145; G16H 40/67

(57) ABSTRACT

The device, process and system of the present invention is made up of a testing device that includes a urine collection vessel with a cap closure that includes a removable circuit chip that conducts a microRNA analysis of existing biomarkers in the collected urine through detection using graphene microchip embedded in the cap closure of the testing device. NFC communication technology is also provided in the circuit chip for non-contact delivery of the testing results to a nearby electronic computer device, such as a smartphone, tablet or other computing device. The present invention provides early detection of ovarian cancer using urine collected from a patient, which has a clear advantage of not being an invasive exam, is easy to collect and easy to analyze and manage contrary to existing procedures, such as blood analysis samples and intra-vaginal exams.

### CROSS-REFERENCE TO RELATED APPLICATION

This application claims the benefit of the U.S. Provisional Application No. 63/484,068 entitled, “DEVICE, METHOD AND SYSTEM FOR DETECTING OVARIAN CANCER” filed on Feb. 9, 2023, which is incorporated herein by reference in its entirety.

### TECHNICAL FIELD

Embodiments relate generally to device, method and system to detect ovarian cancer using urine collected from a patient which has a clear advantage of not being an invasive exam, is easy to collect and easy to analyze.

### STATEMENT REGARDING FEDERALLY SPONSORED RESEARCH

N/A

### BACKGROUND OF THE INVENTION

The present invention relates generally to processes and devices used for the detection of cancer. More specifically, the present invention relates to smart testing devices and associated processes for quickly analyzing and detecting cancers, such as ovarian cancer.

In the United States, for example, 58% women are affected by late ovarian cancer stages. Lack of easy and accessible tests for ovarian cancer detection are impacting the survival rate of women affected by a late prognosis. Worldwide in 2021, 300,000 women were diagnosed with ovarian cancer, out of which 185,000 died. In the US in 2021, 21,000 women were diagnosed with ovarian cancer out of which 14,000 died. Unfortunately, this trend is projected to increase up to 28,000 women diagnosed with 19,000 deaths by 2030 if better and faster screening tools are not available in the next few years. This poses opportunities and challenges that can be overcome thanks to the introduction of new biochemical and sensing technology.

By way of background, gynecologically, ovarian cancer is the type of cancer with the worst survival rate. If diagnosed at stage IV there is a 18.6% of survival after surgery and chemotherapy; survival rate increases up to 34.3% if located at stage III, but still, chemotherapy is needed. Chances of surviving are clearly higher if detected at stage II, up to 69.9%, with a highest survival rate of stage I of 86.4%.

Currently in the prior art, typically, a physician orders a body checkup or orders different tests to assess the health of an individual such as blood cancer antigen CA-125 or intra-vaginal ultrasound. No specific tests are available to remotely detect cancer at the collecting urine and at the same time analyze the data. Some existing techniques exist for home testing but they are restricted to blood testing and right after the patient takes the test, the result still has to be shipped to the address of the laboratory indicated on the shipping label (which typically comes with the test).

Further prior art tests are not desirable if they need to be conducted at the hospital or at a specialized clinic for understanding the result. Often, an individual, due to the present economic and capitalistic landscape, tends to skip routine tests, postponing it when availability is higher. This could cause a woman delays in treatment and a false sense of security.

To date, one of the best approaches, standard of care, is to use intra-vaginal ultrasound to assess whether initial cancer is developing combined with withdrawal of blood to assess the cancer antigen biomarker CA-125.

However, this is clearly an invasive test that requires the woman to make an appointment with the doctor, take time off of work and go through an extremely bothersome procedure.

Regular ultrasonic imaging techniques have long-known benefits. Technological advancements in hand-held probes have been proven to provide better images for soft tissue structures such as the eyes, abdomen, brain, neck, and feet. Conventional techniques for obtaining ultrasound images typically require applying an intra-vaginal probe directly to the patient's body, focusing on the specific area of interest for a particular period of time. The amount of time spent probing is determined by the sonographer's level of experience and by the complexity of the anomaly being investigated.

Unlike CT scans, MRIs, or x-rays, ultrasound imaging technologies are relatively low-cost and do not involve the injection of radiation or contrast. They are also portable. Ultrasound technicians and doctors must constantly balance the quality of scans with the hospitals' needs for efficient, high volume scanning to increase healthcare reimbursements.

The use of hand held probes, however, poses problems that current technology is not yet able to overcome. The evidence is clear that overuse injuries brought on by repetitive muscle stresses associated with the performance of ultrasound exams can lead to muscular damage and, in some cases, career-ending injuries. In addition, the hand-held probe may exacerbate existing pain for patients when technicians apply pressure on the point or area under investigation. This is especially prominent during a prolonged pressure application to a painful area.

Patients may also move during the procedure due to discomfort or pain especially when undergoing intra-vaginal ultrasound, and this can interfere with the quality of the scan, corrupt the ultrasonic reading, or elongate the duration of the exam. If patients are in extreme pain or cannot be still during a scan, the hospital may have to reschedule a session, since efficiency and patient turnover must remain high in the current profit structure.

In order to produce an accurate reading using current ultrasound technology, the system must always have a clear understanding of where the probe is in space. Spatial resolution is often a major obstacle with handheld devices. Accurate readings require the system to have a continuous record of the angular velocity, linear velocity, roll, pitch, and yaw. While current technology offers spherical probe transducers, which concentrate the beam, there is still too much room for human error. For example, if the technician's hand trembles or if there is inconsistency in the amount of pressure they apply, this can compromise the integrity of the reading.

Based on the limitations of current technology, there is a need for more advanced techniques and better systems or procedures. More advanced technology would lead to stronger decision making by physicians and higher quality diagnostic tools. This is especially important because ultrasound technicians and doctors are constantly under the pressure of daily patient turnover and it is critical that the highest possible image quality is available when making high stakes and time-sensitive medical decisions. Additionally, the chances of a missed readings (false negative) at asd high as 25%. Therefore this can entail that the sonographer may miss an initial cancer (too small to be seen on an intra-vaginal ultrasound) which can develop later on into a stage II or stage III ovarian cancer which may become apparent during regular exams (e.g. pelvic examination, imaging and cancer antigen CA-125).

Additionally, when a patient is undergoing a blood withdrawal at a hospital or a clinic, the patient may have to travel long distances or several hours to reach the closest healthcare facility to proceed with the serum cancer antigen analysis CA-125. This forces the patients to take time off of work or even stay overnight if the procedure takes place far from the patient's home.

There is currently a significant amount of scientific research exploring additional techniques such as next generation sequencing, more advanced PCR tests, real-time fluorescence quantitative PCR (qRT-PCR) tests. However, these tests tend to be very expensive and are typically characterized by low sensitivity and specificity (for instance the qRT-PCR test) which limits their routine application. Even though genomic medicine is an ideal target for people affected by cancer, sequencing technologies are limited and cannot take place inside a point of care testing. Next generation sequencing takes about 18 hours on a sequencing machine, but this does not include the time necessary for the analysis and it does not include the throughput time. As soon as the patient's serum is collected by the hospital or the healthcare clinic, the blood is stored at −80 Celsius degree to preserve all the DNA/RNA property and keep it from degradation. After that, the sample has to be sent to a sequencing company which is therefore placed on a waiting list on arrival. The sample is typically processed as soon as the one before is fully sequenced. After sequencing the data, the sample has to go through an alignment process where the ovarian cancer genomic data are aligned with the human genome to see whether there are any specific overlapping matches between the two genomes and confirm an ovarian cancer diagnosis. This step is typically conducted in a lab using special alignment softwares such as STAR Aligner and it typically takes several days as those samples are typically aligned with many other cohorts to keep the alignment expenses down.

Lately, different types of PCR tests based on graphene oxide (GO) have been developed leading to better results in assessing the existence of ovarian cancer at an early stage, this technique is called GO-based qRT-PCR test. However, despite reaching a better sensitivity and specificity in the detection of ovarian cancer, they are still characterized by a long and complex treatment of the sensing surface from a chemical point of view. Typically, graphite powders mixed with sulphuric acid, potassium nitrate and additional compounds have to go through a long and complex chemical preparation.

Due to lack of sensitivity and specificity of both intra-vaginal ultrasound and CA-125 serum test, an accurate diagnosis of ovarian cancer at early stages remains a challenge.

Therefore, there is a particular need for tests of such cancer at earlier stages. There is a further need for smart testing devices and processes that can quickly test for cancers, such as ovarian cancer. There is a need for a testing device, system and process where the testing may be carried out in the patient's home obviating the need to go to a doctor's office. There is yet another need for a testing device to be inexpensive and less costly than in-person blood tests and intra-vaginal examinations. There is a need for a testing device and testing process that is easier and more convenient than known testing methodologies. There is a need for a testing device that is inexpensive to manufacture and simple to use where no batteries are required.

Additional aspects that makes this invention especially useful is the interest in testing as quickly as possible to avoid late prognosis and poor survival rate. People would rather drive to local pharmacies for buying a prescribed tester would suffice, instead of commuting to a hospital or a specialized clinic, which depending on the case, could be far away in terms of commuting time.

Nursing homes would benefit from having the option of conducting urine cancer testing with a fast response instead of having the occupants going to a hospital or special clinics for more invasive testing. Moreover, people living in rural areas, where the average drive for the closest hospitals could be hours away, would also benefit from having a quick diagnosis while maintaining safety standards.

At-risk and undocumented individuals may find these tests available at specialized non-governmental organizations (NGO) testing centers or at local trustworthy institutions as the test respects anonymity standards.

Pharmacists and professionals will benefit from the introduction of this methodology as it will save time for much more time-consuming tests such as blood analysis or calculation of treatment and drug allocation for a specific patient.

The testing device, system and process of the present invention is also environmentally friendly as the smart lid can be recycled and reprogrammed for multiple uses benefiting the environment.

### SUMMARY OF THE INVENTION

The testing device, system and process provides a testing methodology that is superior to known and existing testing devices systems and processes.

The device, process and system of the present invention is made up of a testing device that includes a urine collection vessel with a cap closure that includes an optionally removable integrated circuit chip that conducts a microRNA analysis of existing biomarkers in the collected urine through detection using graphene sensors embedded in the cap closure of the testing device. NFC communication technology is also provided in the circuit chip for non-contact transmission of the testing results to a nearby electronic computer device, such as a smartphone, tablet or other computing device. Thus, the present invention provides urine analysis, which has a clear advantage of not being an invasive exam, is easy to collect and easy to analyze and manage contrary to existing procedures, such as blood analysis samples and intra-vaginal exams.

In view of the foregoing, an embodiment of the present invention is to provide a new testing device that can detect cancer at its earlier stages using only urine from the patient.

A further embodiment of the present invention is to provide a smart testing device and process that can quickly test for cancers, such as ovarian cancer.

Yet another embodiment of the present invention is to provide a testing device, system and process where the testing may be carried out in the patient's home obviating the need to go to a doctor's office, healthcare clinic or hospital.

Another embodiment of the present invention is to provide a testing device that is inexpensive and less costly than in-person blood tests and intra-vaginal examinations.

A further embodiment of the present invention is to provide a testing device and testing process that is easier and more convenient than known testing methodologies.

Further, another embodiment of the present invention is to provide a testing device that is inexpensive to manufacture and simple to use where no batteries are required.

Additional features and advantages of the various aspects of the present invention become apparent from the following description of its preferred embodiments. The description provided should be taken in consideration with the accompanying drawings. The aforementioned embodiments do not represent the full scope of the invention. The references following the description of the preferred embodiments are made thereof to the claims and herein for interpreting the scope of the present invention.

### BRIEF DESCRIPTION OF THE DRAWING FIGURES

The novel features that are characteristic of the present invention are set forth in the appended claims. However, the invention's preferred embodiments, together with further objects and attendant advantages, will be best understood by reference to the following detailed description taken in connection with the accompanying Figures:

FIG. 1 is an overview of the sensor holder board and the communication board where is possible to see the front, side and back view of every board;

FIG. 2 the sensor holder board and the communication board where is possible to see the front, side and back view of every board, with an additional particular that shows the graphene microchip and the graphene array matrix;

FIG. 3 shows how the sensor board and the communication board is mounted underneath the smart lid and an overall image of the smart testing device;

FIG. 4 shows an exploded side view version of the testing device where is possible to see all the components and the way they are assembled;

FIG. 5 shows an exploded frontal view version of the testing device where is possible to see all the components and the way they are assembled;

FIG. 6 shows a flow diagram of the data path process of the present invention where the testing kit is provided to the patient by a pharmacy or other medical or healthcare institution;

FIG. 7 shows a flow diagram of the data path process of the present invention where the testing kit is provided to the patient directly by mail; and

FIG. 8 shows the general workflow in accordance with the present invention passing through the different phases from using the testing device of the present invention, operating the NFC detection with an electronic computer device, such as a smartphone, tablet or other computing device, sending the results to the primary physician and, if necessary, to the healthcare provider.

FIG. 9 shows a typical Dirac voltage curve for void (device system not used but calibrated), healthy detection and diseased detection for microRNA miR-21 (ovarian cancer)

FIG. 10 shows an enlargement of FIG. 9 where it is possible to see the voltage shift of the void vs healthy vs diseased. As expected the diseased shift in comparison to the healthy is higher. Meaning that the concentration of the microRNA detected on the surface of the GFET is higher than the healthy causing an increased output voltage and consequently an increased voltage shift. In the same image it is also shown the delta h (for healthy) and the delta d (for diseased).

FIG. 11 shows the testing device of the present invention while connected via NFC to the mobile application in the process of analyzing the content of the urine. The image shows an idealized graphical user interface of the device and the steps that the user needs to follow to arrive to send the results to the curing doctor or provider.

FIG. 12 shows the mobile application containing the information transferred from the testing device of the present invention. The data are visible only after being analyzed by the doctor or the healthcare provider. It is possible to see the microRNA concentration content analyzed. An idealized graphical user interface on how the data are shown to the user is therefore provided for explanation purposes.

FIG. 13 shows a PCA graph showing the difference between healthy patients and patients with overexpression of microRNA miR21.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skills in the art to which this invention belongs. For any apparent discrepancy between the meaning of a term in the art and a definition provided in this specification, the meaning provided in this specification shall prevail.

“Base R” is an R-based computer program.

“Dirac Voltage” has the electronic art-defined meaning. The Dirac Voltage of a GFET is defined as the gate bias that sets the charge neutrality condition in the graphene channel, namely, the gate bias that results in a minimum conductivity.

“FET” has the electronic art-defining meaning. The field-effect transistor (FET) is a type of transistor that uses an electric field to control the flow of current in a semiconductor.

“GFET” has the electronic art-defined meaning. The GFET is defined as a Graphene Field Effect Transistor and it is mainly composed of a graphene channel between two electrodes with a gate contact that modulates the response of the channel electronically.

“Next Generation Sequencing (NGS)” has the molecular biological art-defined meaning. NGS technology is typically characterized by being highly scalable, allowing the entire genome to be sequenced at once. Usually, this is accomplished by fragmenting the genome into small pieces, randomly sampling for a fragment, and sequencing it using one of a variety of technologies.

“NFC” has the electronic art-defined meaning. Near-Field-Communication (NFC) is a short-range wireless connectivity technology that uses magnetic field induction to enable communication between devices.

“Principal Component Analysis (PCA)” has the computer-art and molecular biological art-defined meaning. Principal component analysis is a statistical procedure that uses an orthogonal transformation to convert a set of observations of possibly correlated variables (entities each of which takes on various numerical values) into a set of values of linearly uncorrelated variables called principal components.

“STAR aligner” is the Spliced Transcripts Alignment to a Reference (STAR), a fast RNA-seq read mapper, with support for splice-junction and fusion read detection. STAR aligns reads by finding the Maximal Mappable Prefix (MMP) hits between reads (or read pairs) and the genome, using a Suffix Array index. Different parts of a read can be mapped to different genomic positions, corresponding to splicing or RNA-fusions. The genome index includes known splice-junctions from annotated gene models, allowing for sensitive detection of spliced reads. STAR performs local alignment, automatically soft clipping ends of reads with high mismatches. Dobin et al., “STAR: Ultrafast universal RNA-seq aligner.” Bioinformatics, 29(1), 15-21 (January 2013).

The new and unique smart testing device, process and system of the present invention is discussed in detail below. The present invention relates to systems and methods for detecting ovarian cancer in women at the initial stage through the analysis of specific ovarian cancer biomarkers using a smart urine tester. The user only needs an electronic computer device, such as a smartphone, tablet or other computing device in addition to the device of the present invention to conduct such a test.

The present invention is a smart testing device that quickly analyzes and detects ovarian cancer in women at the initial/early stage. The device, system and process of the present invention is a touchless system where no battery is required as it is based on near field communication (NFC) technology, which is non-contact in nature. Thus, only the tester device of the present invention and an electronic computer device, such as a smartphone, tablet or other computing device are needed to conduct the test.

By way of background, a complete blood count is a test that checks the levels of red blood cells, white blood cells and platelets. Blood may also be tested to see how well the kidneys or liver are working. Urine may be tested to look for any abnormal cells or bleeding that may be coming from the bladder or kidneys. In some cases, blood and urine may also be tested for a protein that might help diagnose a blood cancer called myeloma. Blood and urine tests can be used to check general health, and can also provide information about whether a cancer of unknown primary (CUP) may have started.

Tumor markers are chemicals made by some cancer cells, and high levels are found in the blood, urine or other body fluids of some people with cancer.

For example, markers that may suggest certain types of cancer include: a) prostate specific antigen (PSA)—prostate cancer; b) alpha-fetoprotein (AFP)—testicular and liver cancers; c) human chorionic gonadotropin (HCG)—testicular cancer; d) carcinoembryonic antigen (CEA)—bowel, lung, pancreatic, stomach, ovarian, breast, thyroid and liver cancers; c) cancer antigen 125 (CA125)—ovarian, endometrial, fallopian tube and peritoneal cancers; f) cancer antigen 19-9 (CA 19-9)—pancreatic, stomach, bile duct, gall bladder and ovarian cancers and g) cancer antigen 15-3 (CA15-3)—breast cancer

In the past few years, carbon materials, such as graphite, carbon nanotubes, and, especially, graphene, have been considered promising materials for biosensing applications due to their excellent physical properties such as stability, electrical conductivity, thermal properties, low cost, high elasticity and flexibility. Additional properties that make the use of graphene for this invention the perfect material are its strong antibacterial effect, meaning that bacteria will not grow on a graphene surface, low electricity consumption and low Joule effect and its ability to generate electricity by exposure to sunlight. Graphene is a well known 2D material composed of a single layer of carbon atoms, tightly bound in a hexagonal lattice form and it possesses a unique sp2-hybridized crystal structure. The great benefit of graphene materials for microRNA detection is that it can be functionally modified by DNA/RNA or other functional nanomaterials which makes graphene an ideal platform for nucleic acid detection.

To truly and effectively treat microRNA as a useful and reliable biomarker tool for early stage ovarian cancer it is important to develop sensing technology that is able to quantitatively measure the expression level of specific microRNA. Additionally, due to the existence of known problems such as low abundance and quick and easy degradation of microRNA it is of high importance that the tester detect concentration of microRNA as efficiently as possible together with the slightest concentration change between control (non diseased) and diseased sample.

Any integrated circuit may be used in the present invention to provide the appropriate sensors to detect the desired biomarkers. For example, the sensor chip provides an output voltage that is digitized and detected by the patient's smartphone using NFC, as described above. Then, the patient's smartphone parses the data received to display the test results. Also, it is possible that the data received from the sensor chip is first sent to an external server, preferably in an encrypted manner, for processing of the received data to determine the test result.

The abovementioned integrated circuit is composed of graphene field technology or also called GFET. A GFET microchip has the great advantage of being very sensitive to micoRNA. In particular a GFET microchip can be used for the early detection signs of ovarian cancer conducting a simple urinary test.

The test employed by the present invention is of a type that analyzes microRNA, which modulates gene expression in different cancer types. MicroRNA analysis is a powerful tool that can be used to differentiate health and cancerous tissues and therefore understand the population that might be subjected to early stage cancer development. The device, system and process of the present invention is based on microRNA analysis of existing biomarkers using GFET mounted on a PCB board and embedded in the lid of the tester device of the present invention, also called a smart lid. The smart lid is the device operating the biomarker detection.

The present invention provides the opportunity to potentially decrease cancer mortality in women affected by all stages, but specifically with later prognosis, which typically is connected to poor survival rate. In addition, it is a non-invasive approach which is more favorable.

Specific biomarkers that can inform about the occurrence of ovarian cancer and whether or not a cancer is developing at the initial stage are described below. The device of this invention can also be used as a surveillance system for people already affected by ovarian cancer and can be informative on the status of the disease on a specific patient. In case of administration of a drug, if the drug is effective it shall be related to the decrease of certain biomarker, while if the drug is not having any effect or improving the condition of the patient in any meaningful way, then the biomarker level should either remain the same (if a previous test was executed) or increase, in case of a progression of the disease.

MicroRNA are a class of short non-coding single-stranded RNAs (approximately between 18 and 24 nucleotide length) that bind to the 3′-untranslated regions (also known as 3′-UTR). MicroRNA have been often found and attributed as a major cause of developmental processes such as cell division and cell proliferation, apoptosis and cancer.

The specific expression profile of microRNA changes with developmental stages, pathological conditions and dysregulation of cellular functions. Notably, the most significant miRNAs with abnormal expression levels (both upregulated and downregulated) associated with ovarian cancer are: Let-7, miR-200, miR-21, miR-214, miR-146a, miR-106b, miR-20a, miR-210 and miR-19a, miR-143, miR34a, miR34b; miR34c, miR-125, miR-199a and miR-30a-5p.

Specific microRNA typically appear at each specific stage of the progression of the disease and are described below.

Initially, the detection for down-regulated miRNA are Let-7 which can target HMGA2, IMP1, Mlin-41 and miR-200 which can target ZEB1 and ZEB2.

Additionally, very important is the testing of the well known upregulated ovarian cancer microRNA biomarkers such as: miR-21, miR-214, Let-7, miR-200, miR-146a, miR-106b, miR-20a, miR-210 and miR-19a. Which will complement the analysis. The main reason for those would be that miR-21 and miR-214 will target PTEN, Let-7 will target HMGA2, miR-200 will target ZEB1, miR-146a will target BRCA1 and miR-106b and miR-20a will target p21.

Other ovarian cancer biomarkers downregulated that can support the detection can also be miR-143 which can target K-Ras; miR34a and miR34c; miR34b which can target c-Met, Notch and Cdk6. Finally miR-125 which can target ERBB2.

During an uncontrolled cell proliferation and abnormal apoptosis the following microRNA can be detected in the upregulation phase: miR-210 which can target E2F3; miR19a which can target TSP1; for the downregulation phase it is possible to detect miR-9 which can target NFKB1; miR-199a which can target IKKB and Cox-2.

During angiogenesis and inflammation in the downregulated phase it is important to identify miR-200 which can target ZEB1 and ZEB2.

During transcoelomic metastasis, which process is upregulated, it is important to identify miR-200 which can target ZEB1 and ZEB2.

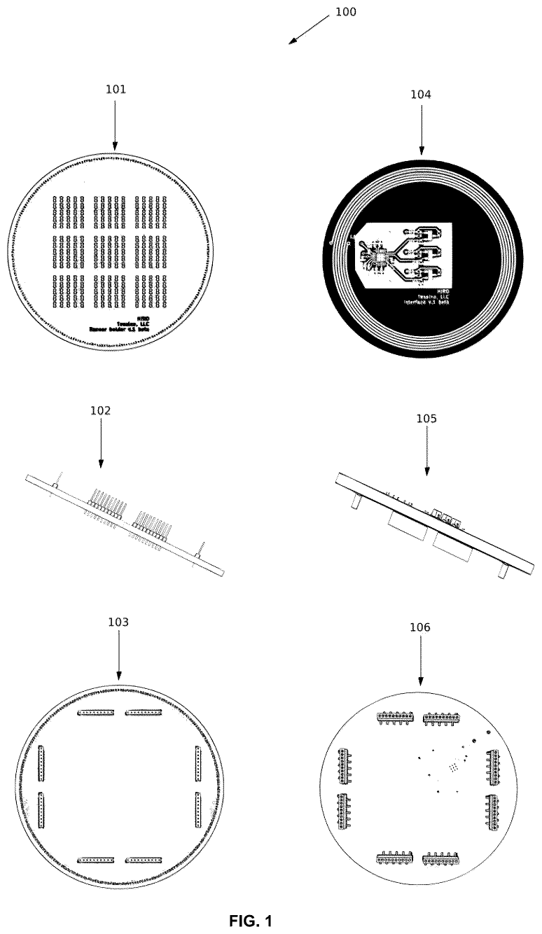

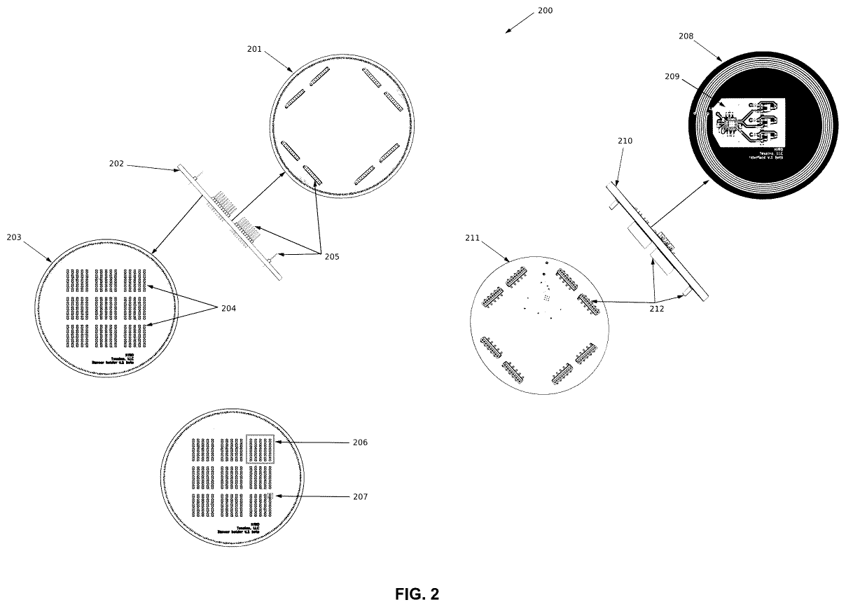

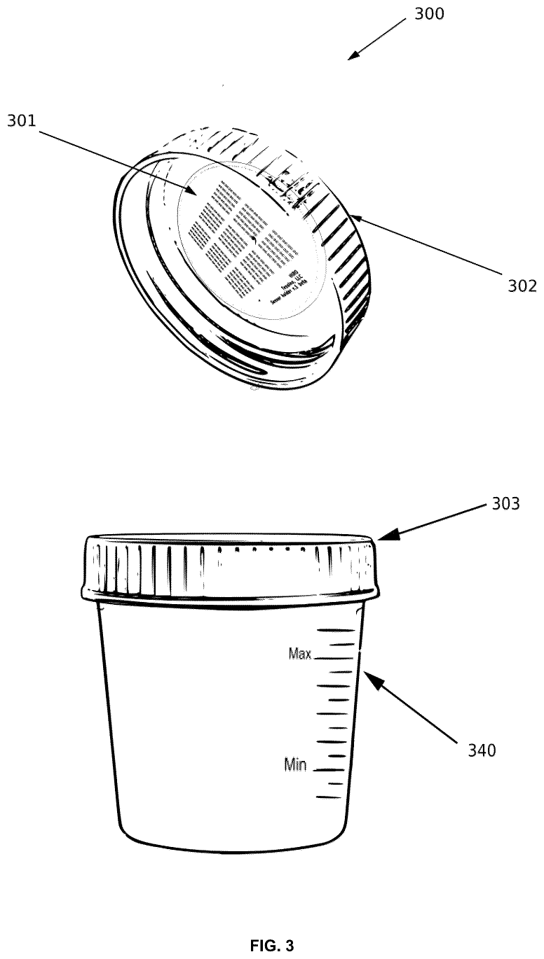

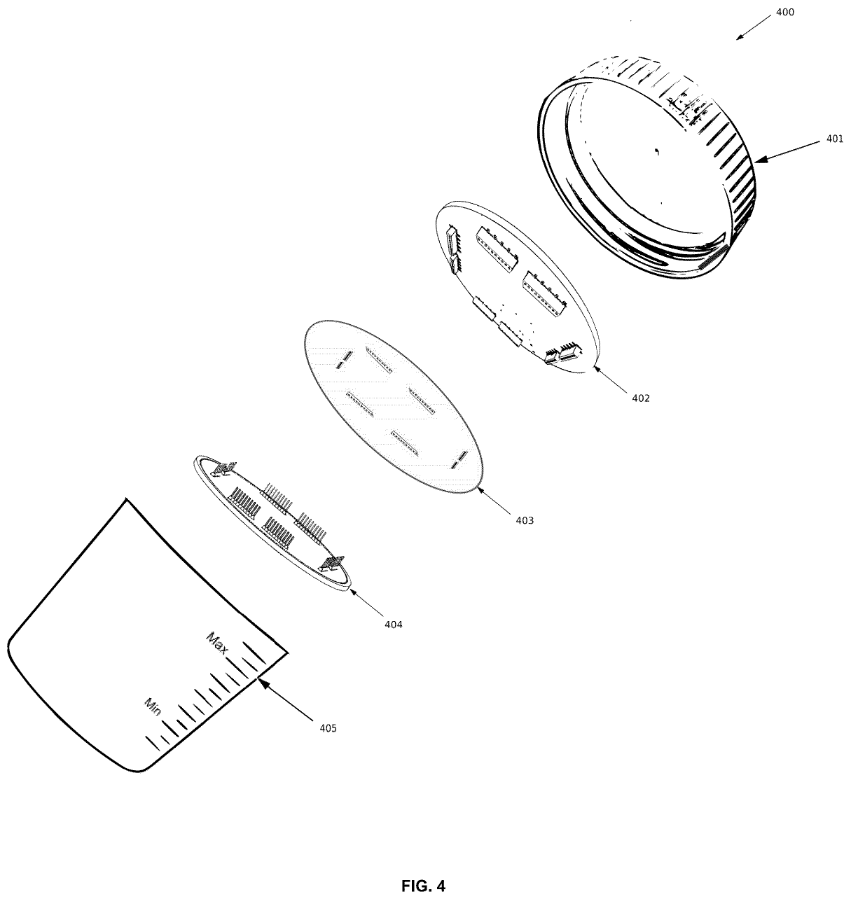

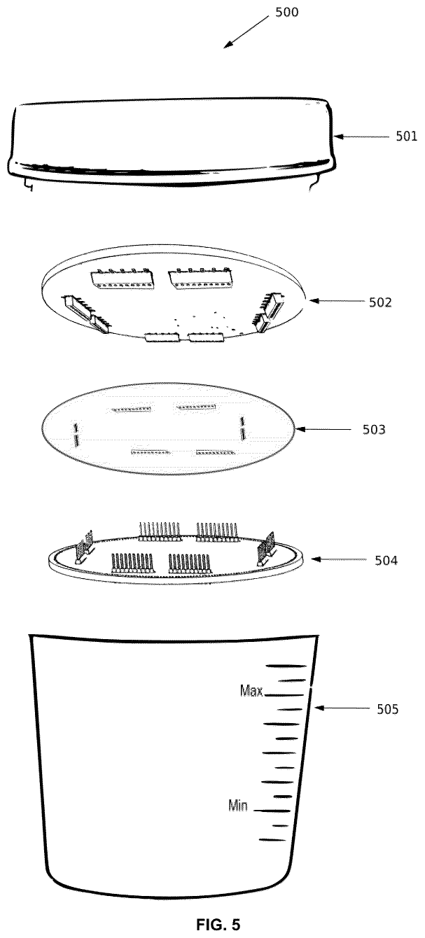

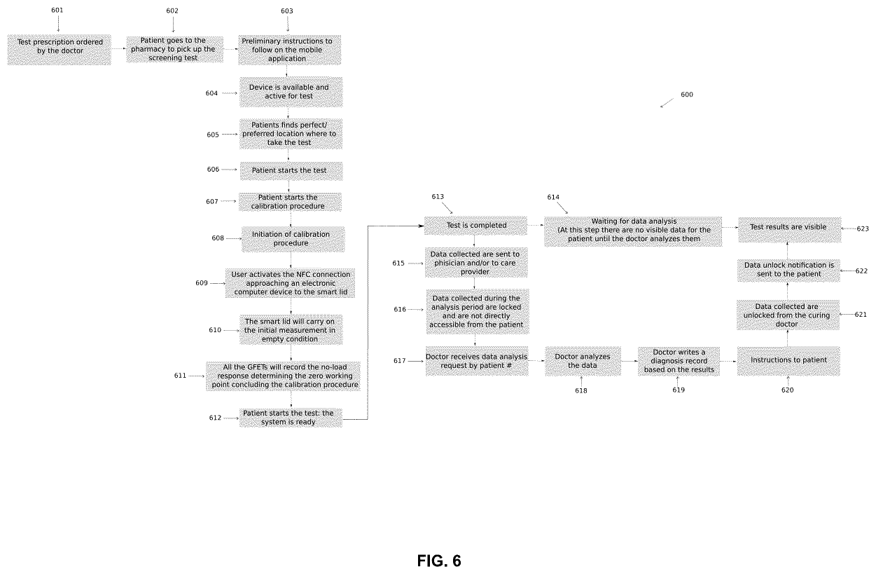

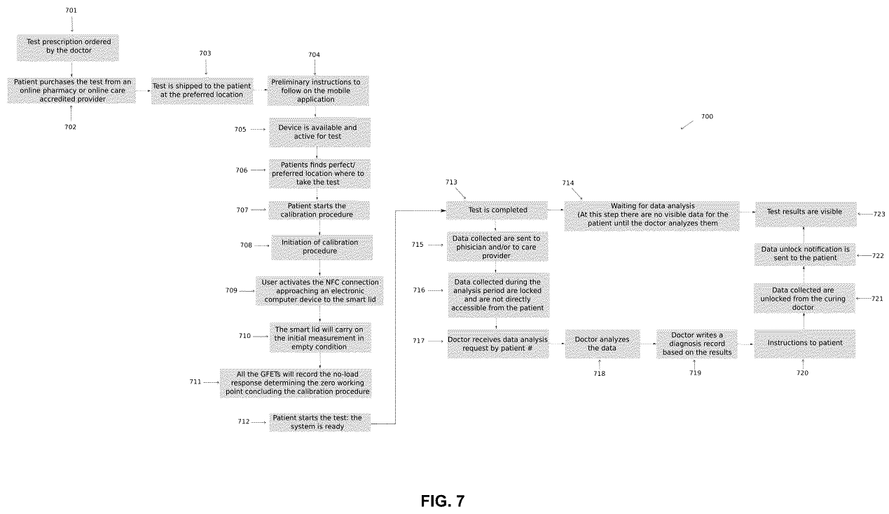

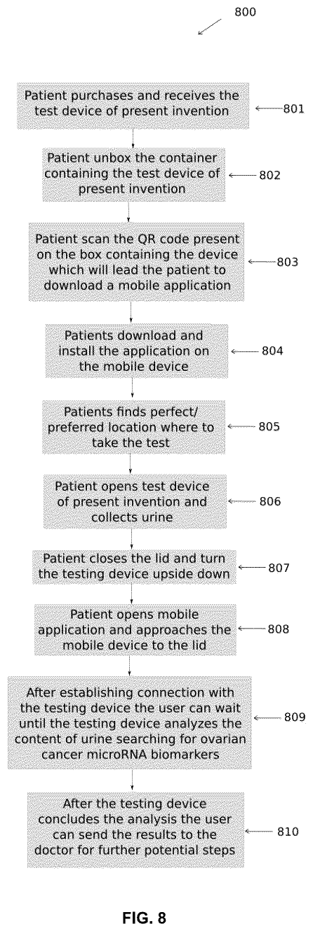

As shown in the attached drawing FIG. 1, FIG. 2, FIG. 3, FIG. 4, FIG. 5, FIG. 6, FIG. 7 and FIG. 8, the tester device and the general workflow is shown. The present invention is environmentally friendly with the possibility of the entire detection kit being disposable.

As shown in FIG. 1 and FIG. 2, the device of the present invention is constructed from simple consumable electronics, where Graphene Field Effect Transistor (GFET) sensors are assembled on a PCB board and mounted inside of a smart lid (or closure cap) of the device attached to a liquid vessel. Given the simple and intuitive nature of the test, it is possible to use the test for additional purposes such as: i) a regular urine test, b) drug test, c) breast cancer, d) other types of cancer or any other disease. The GFET are configured to and arranged on an integrated circuit sensor (PCB board) and attached to the inside part of the cap as shown in the exploded drawing of FIG. 3.

The testing device is composed of: a sensor holder board (front view is 101 and back view is 103) where the GFET are mounted, a communication board (front view is 104 and back view is 106) that communicates with an antenna via NFC with an electronic computer device, such as a smartphone, tablet or other computing device, and a waterproof layer (shown on 403 or 503) with existing holes physically connecting the sensor holder board 103 and the communication board 106. The connection between the two boards happens via pin headers 205 (as shown in FIG. 2) which will be inserted inside the sockets 212 present on the communication board. The pin headers 205 on the sensor holder board, will therefore pass through the waterproof layer 403 or 503 and will connect to the sockets 212 of the communication board. In FIG. 2 it is possible to see a particular sensor holder board carrying the GFET. 206 shows a GFET array mounted as a matrix, while 207 shows a single GFET. The detection happens using an array of GFET (mounted as a matrix in 206). Therefore, every array will measure the concentration of a specific biomarker. As a way of example, if a patient needs to measure the concentration of 9 specific biomarkers, the testing device will have to have a total of 9 GFET arrays (9 different matrices of GFETs). Multiple GFET on a single matrix (meaning rows and columns of the matrix) make sure the measurement is precise. Clearly the only board that has to be in contact with the urine is the sensor holder board front view 101 (or 203). The waterproof layer will make sure that no liquid will reach the communication board. The communication board can therefore remain dry and proceed with NFC connection to an electronic computer device, such as a smartphone, tablet or other computing device. Finally the system sensor holder board, waterproof layer, and communication board are screwed in the bottom part of the lid as shown in the drawings attached. The waterproof layer will make sure that no liquid will ever be in contact with the communication board so that the communication board remains fully dry.

As soon as the liquid is in contact with the GFET, the specific matrix in charge of capturing the specific microRNA target will start the detection. The more the concentration of the specific biomarker is detected on the surface of the GFET, the higher the electrical response for that specific GFET array. Consequently, the lower the concentration of the specific biomarker on the surface of the GFET, the lower the electrical response from the GFET matrix.

The voltage output signal is therefore passed to the graphical user interface of an electronic computer device, such as a smartphone, tablet or other computing device. This result is however not initially visible from the patient as it needs to be screened by the doctor first.

A single smart lid can carry several sensors but are physically separated to increase the efficiency and isolation of the electronic measurement. However, multiple sensors can not be mounted on the same circuit because the overall efficiency will decay, but if the sensors are mounted as a stand-alone sensor the efficiency can be considered a single value. Therefore the comprehensive efficiency of the sensors is higher.

Based on a specific biomarker detected, the difference between the detected concentration (expressed as an output voltage) versus the concentration on control (non-disease) condition will inform the doctor about the result of the test and whether additional steps need to be taken in considerations. The different voltage output between the detected voltage (concentration of microRNA on the surface of the GFET array) and the control (no disease) will inform the doctor about the current status of the disease (if any is detected).

Initial calibration and instantiation of the zero point condition: Two operating steps are required by the user in order to properly conduct the measurement: a) the user activates the NFC connection approaching an electronic computer device, such as a smartphone, tablet or other computing device. The smart lid will therefore carry on the initial measurements in empty conditions. All the GFETs will record the no-load response determining the zero working point. Basically this is just a calibration step with no content to analyze in the urine vessel.

b) Once the smart lid notifies the “system ready”, the user can proceed by opening the vessel, filling the testing device with a urinary sample, closing the vessel with the smart lid and turning the test upside down so the smart lid can analyze the content.

How is the measurement done: In transistors that exploit the Field Effect Transistor (FET) field effect principle, the current flow between the drain and source terminals is modulated by applying a potential difference to the gate terminal. An increase in current flow occurs only if this potential difference exceeds a certain value called threshold voltage. Below the threshold voltage value the transistor does not conduct and is said to be cut off or off. Whereas, in transistors whose channel is made of graphene (GFET) such as the present invention, due to the characteristic molecular structure of this material, the increase in current flow occurs for gate voltage values both higher and lower than the threshold voltage (ambipolar operation), and at this voltage the current has a minimum almost close to OA (Ampere). In GFETs this potential is called Dirac voltage or Dirac point. The interaction of some types of organic molecules or species such as microRNA with graphene causes a variation in the conductivity of this material which also causes a variation in the Dirac voltage. The measurement of this variation allows us to determine the extent of the chemical interaction of microRNA or other substances using GFET.

In particular, it was observed that trapping on the graphene channel of the GFETs some of the markers in the presence of tumor (such as for instance the microRNA miR-21 provided also in the drawings) determines an appreciable and therefore measurable shift in the Dirac voltage as high as 4%. The device of the present invention is therefore able to detect and pick up the Dirac voltage shift of the measurement. For example, the patients with a 4% Dirac voltage shift with respect to the control (healthy) is the type of shift that our smart test lid device is able to pick up during the microRNA measurement concentration phase. The GFET provides an output voltage that is digitized and detected by the patient's electronic computer device, such as a smartphone, tablet or other computing device as described above. As soon as the measurement is complete, the smart lid will emit an acoustic signal alerting the patient of the concluded test. Then, the patient's electronic computer device parses the data received to display the test results only after being examined by a doctor. The data sent from the patient's electronic computer device, such as a smartphone, tablet or other computing device is first sent to an external server, preferably in an encrypted manner, for processing of the received data to determine the test result. The data is analyzed by the doctor. The doctor prepares the diagnosis and informs the patient about the final result. After that the doctor makes the data available to the patient on the patient's electronic computer device, such as a smartphone, tablet or other computing device for visualization purposes.

How is the comparison made to differentiate diseased from healthy; Each GFET or array of GFETs functionalized and electrochemically sensitive to a specific microRNA marker, presents a variation of the Dirac voltage in the presence of that particular marker and to an extent correlated to its concentration on the graphene surface. The response of the GFET to each marker of interest is experimentally evaluated a priori to obtain the concentration of the markers they express for healthy subjects and for diseased subjects and the variation in the electrical quantity of the GFETs used for the measurement. The optimal threshold values of this quantity are thus defined which discriminate between the two groups (healthy and diseased); the related thresholds for each marker are stored in a centralized database available to the doctor when analyzing and writing the final diagnosis. Therefore, the Dirac voltage for a specific biomarker for healthy patients is known before conducting the test with the present invention. The device measures the characteristic electrical quantity (i.e. voltage, current and voltage shift between healthy and current measurement). The measurement results completed with the present invention are transmitted to the doctor via an electronic computer device, such as a smartphone, tablet or other computing device. The results are processed by the doctor which compares them with the existing profiles and threshold, determining whether the measurement relates to a sample of a healthy or diseased subject. Healthy concentrations of microRNA are available in existing literature.

FIG. 6, FIG. 7 and FIG. 8 are showing the data process flow diagram from when the urine tester device is ordered to when the doctor can see the final result of the analysis. In FIG. 6, the testing device is picked up at the pharmacy after being ordered by the curing physician. The pharmacists scan the urine testing device of the present invention and assign the device (which will come properly packaged) to the patient. The package of the device will contain a specific QR code (or alternatively a bar code) which is linked to that specific device. The pharmacist will also activate the testing device and assign a specific type of test to the patient according to the doctor's order. For example if the patient has to conduct an “Ovarian Cancer Screening” the pharmacist will set the testing device for “Ovarian Cancer Screening” and set the timing of the analysis on the NFC sensor of a total of, for example, 30 minutes necessary to run the screening.

The process flow diagram of FIG. 6 starts with block 601, that is when the test is prescribed by the doctor. The doctor may prescribe it because of family history of cancer, for surveillance reasons or because the patient needs pre and post surgery screening. In particular for post-surgery screening the tester can act as an efficient surveillance tool. The patient goes to the pharmacy on block 602 and picks up the tester system. On block 603 a patient needs to follow the preliminary instructions of use of the device in order to install the mobile application so that the device is ready to be used at any moment, as shown in block 604, whenever the patient is more comfortable, block 605. When ready, as shown in 608, the patient can start the calibration process. Therefore, the patient activates the NFC connection approaching an electronic computer device to the smart lid. After this, the smart lid will carry on the initial measurement in empty condition as shown in 610. Finally all the GFETs will record the no-load response determining the zero working point concluding the calibration procedure as shown in 611.

The patient may start the test and collect the urine in the tester and the screening process starts as shown in block 612. After, for example, 30 minutes the test is completed as shown on block 613 and 615 shows that all the data collected by the tester are sent to the physician or the care provider (or both). At this point the mobile application is locked at 616 and no more actions are needed from the patient side, 614. As soon as the data are transferred to the doctor, typically through a receiving computer application, the doctor receives a data analysis request number from a specific patient, which carries the first and last name of the patient together with the type of test that was ordered as shown un 617. At this stage in 618, the doctor can go through the results and write a diagnosis based on the result of the test, 619, and the patient is informed about the result of the test, 620, before visualizing the data. The doctor at this point can unlock the data as shown in 621, and notify through text message or email or an on-line chat on the mobile application as in 622. Finally, the patient is able to see the result of the test as shown in the last block 623.

FIG. 7 shows almost the same process explained on FIG. 6 with the main exception that the same test can be purchased by the patient on an online pharmacy or an accredited healthcare provider after presenting the prescription of the doctor as shown in block 702. After the patient receives the test at the preferred location 703, the processing route of the operations is the same as FIG. 6.

The operations following operating the device and the collection of the urine are properly explained in FIG. 8 in the flow diagram 800 where the patient purchases and receives the testing device of the present invention at a preferred location, 801.

The patient can unbox the container, 802, containing the test device of the present invention paying attention to the QR code (or alternatively a bar code) that is printed on the outside packaging of the box as in 803 which is followed by the installation of the testing application on an electronic computer device, such as a smartphone, tablet or other computing device, 804. When at a preferred location 805, the patient starts the calibration step previously described, after that, the patient in 806 can unscrew the lester lid of the device and proceed with collecting urine until the liquid level reaches a specific minimum indicator clearly visible on the container. At that point the patient can close the tester lid and turn the test device of the present invention upside down as in 807. After that the patient can launch the mobile application and can approach the mobile device to the tester lid, as in 808. After establishing an NFC secure connection with the testing device, the user can wait until the testing device analyzes the content of the urine collected searching for ovarian cancer microRNA biomarkers, as shown in 809. In the meantime the patient can go back to regular activities. As soon as the tester system finishes the analysis of the content a sound will alert the patient that the analysis is concluded and the patient can approach again the mobile device where the application was installed, to re-establish a secure NFC connection and send the results to the doctor and wait for further instruction on the final diagnosis as shown in block 810.

The mobile application described in the present patent, it is a simple application which can be coded using any programming language such as HTML, CSS or JavaScript. The core innovation of the present patent resides in the fact that analysis of the content of the urine is done by the smart lid. The detection is mainly due to the calculation of the concentration of the specific biomarkers on the graphene sensor area and the higher the concentration of the biomarker, the higher is the output voltage recorded by the tester lid. The intensity of the voltage, (and current) is then translated into a readable (digitized) number which is the representation of the calculated concentration, which is the voltage shift between the measured and existing values.

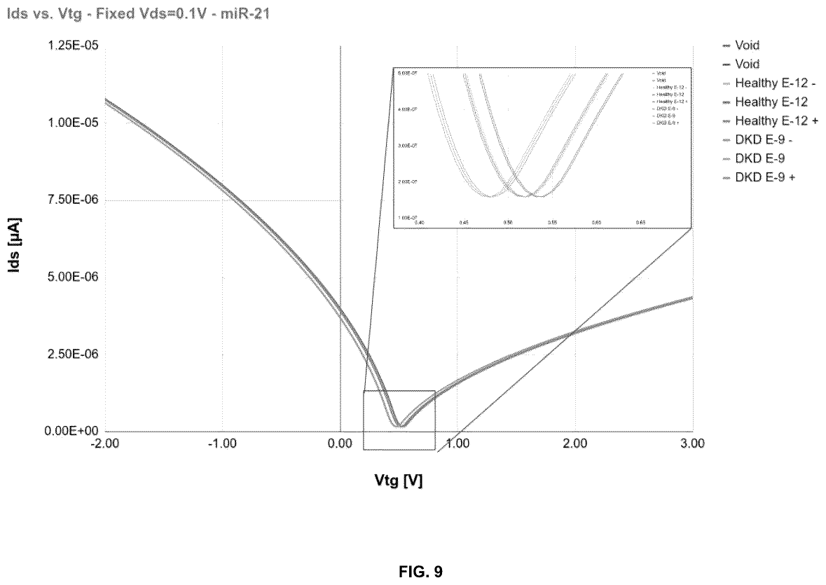

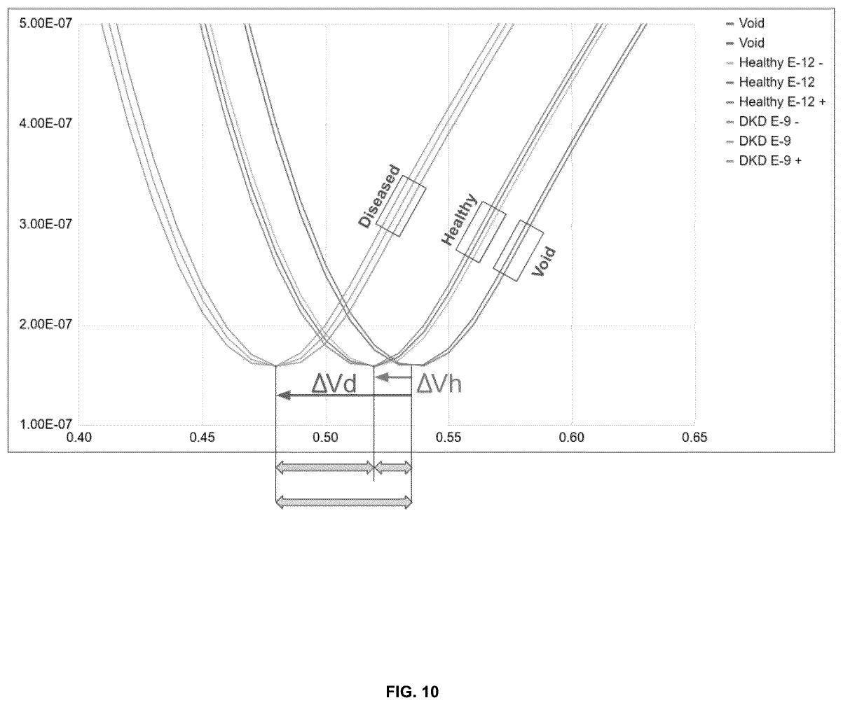

FIG. 9 and FIG. 10 are a representation of the voltage shift of the microRNA miR-21, which is also one of the microRNA found to be upregulated in the presence of ovarian cancer. The drawing on FIG. 9 shows a voltage shift between void samples (calibration phase), and three different measurements for healthy samples and diseased samples. The graph is expressed in voltage versus current and an enlargement of the minimum point is shown in FIG. 10. What is important to observe in FIG. 10 is the voltage shift between the samples. Notably, the void, healthy and diseased tends to cluster together. Therefore, the variation in voltage (ΔVh) for the healthy in comparison to the void versus the variation voltage (ΔVd) for the diseased in comparison to the void will be at least 4% or higher.

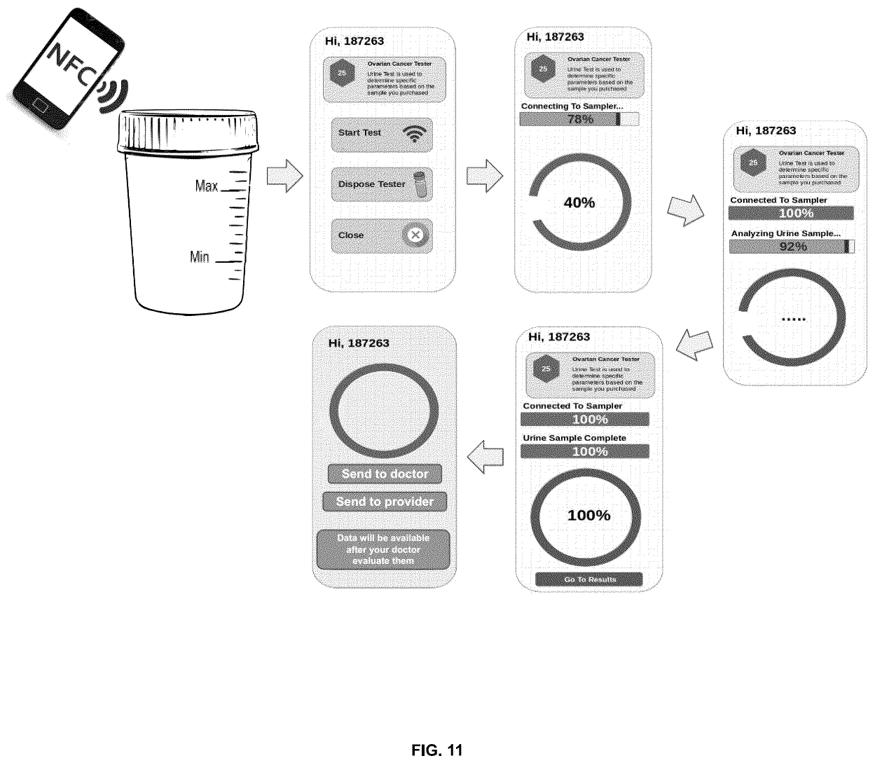

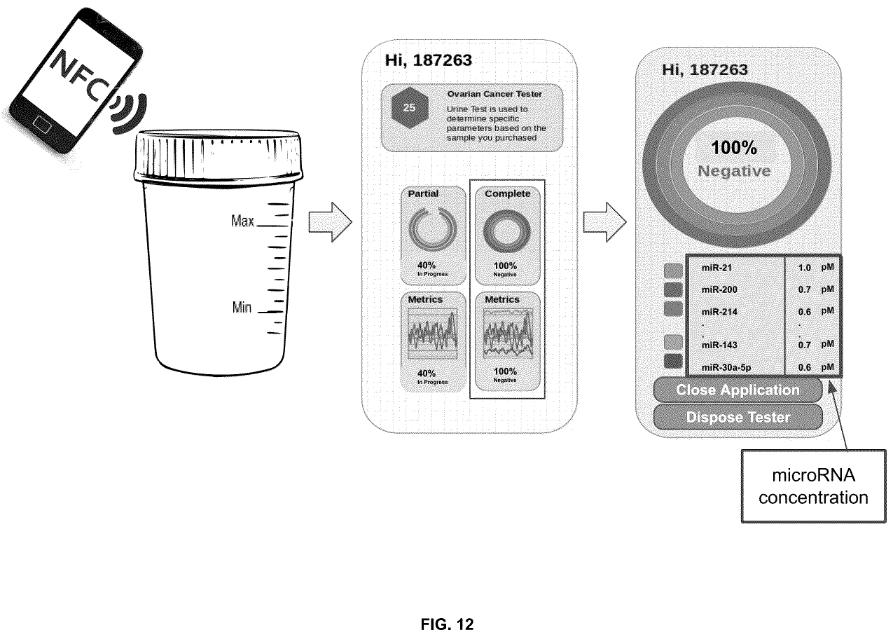

FIG. 11 and FIG. 12 are a representation of the complete cycle where it is shown the testing device of the present invention which is approached by a nearby electronic computer device, such as a smartphone, tablet or other computing device to establish a secure NFC connection. As soon as the connection is established an idealized graphical user interface will prompt the user, through seeing different graphical user interface pages: a) when the connection with the smart lid is established; b) when the tester is ready to proceed with the urine analysis (therefore the calibration phase is concluded); c) when the tester has started the analysis of the urine sample; d) when the analysis is complete and e) when to send the result to the doctor. FIG. 12 shows the results after the doctor analyzes the results and completes the diagnosis. An idealized graphical user interface with the completed results is also visible. The interface will show the type of microRNA detected and the concentration for that specific microRNA.

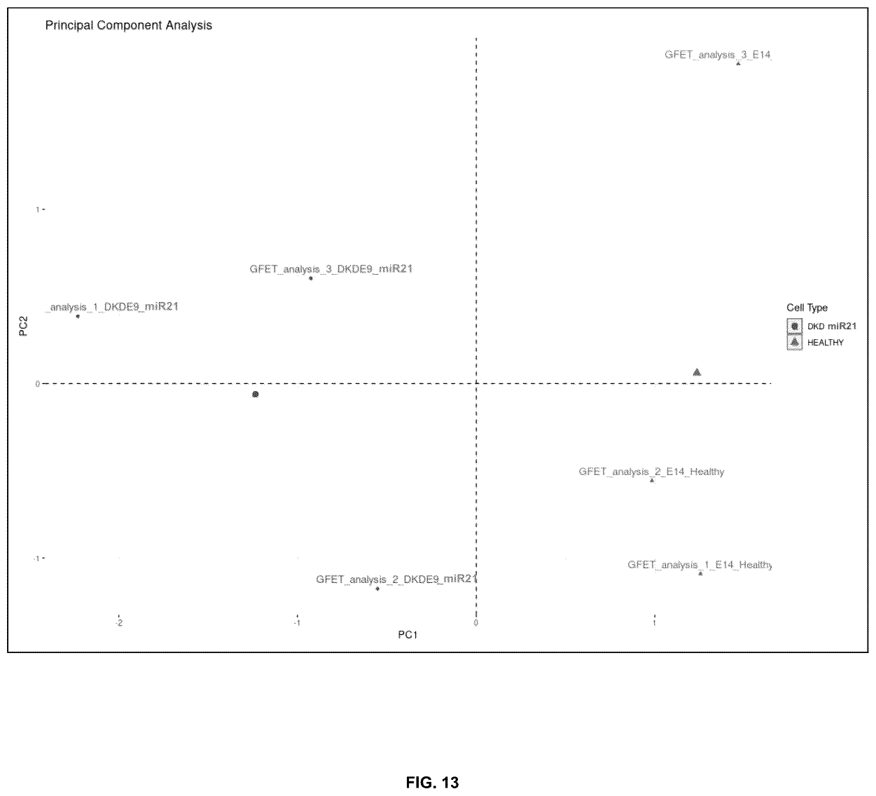

Finally on FIG. 13 it is depicted through the use of a PCA analysis the output between health samples and diseased samples, expressing a higher expression of microRNA miR21. The PCA diagram is able to precisely distinguish the healthy population in comparison to the population affected by overexpression of miR21.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

It would be appreciated by those skilled in the art that various changes and modifications can be made to the illustrated embodiments without departing from the spirit of the present invention. All such modifications and changes are intended to be within the scope of the present invention except as limited by the scope of the appended exemplary claims.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

### LIST OF EMBODIMENTS AND EXAMPLES

Specific systems and methods of obtaining laser ultrasound images completely contactless have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as those skilled in the art will recognize. When the specification or claims recite methods, steps or functions in order, alternative embodiments may perform the tasks in a different order or substantially concurrently. The inventive subject matter is not to be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by one of ordinary skills in the art to which this invention belongs. This invention is not limited to the particular methodology, systems, protocols, and the like described in this specification and, as such, can vary in practice. The terminology used in this specification is not intended to limit the scope of the invention, which is defined solely by the claims.

What is claimed is:

1. A device for the detection of cancer, comprising:
a. a vessel for containing urine having a top open end;
b. a cap, having an inner surface, attachable to the top open end of the vessel;
c. an electronic sensor holder board containing GFET arrays and pin headers attached to the inner surface of the cap;
d. an electronic communication board containing a control circuit and sockets;
e. a waterproof layer with existing holes connecting the sensor holder board pin headers and the communication board sockets;
f. an electronics GFET array configured to conduct an electronic microRNA concentration analysis of cancer biomarkers in the collected urine and;
g. the communication board further including a communication module to transfer microRNA analysis results generated by the microRNA sensor holder board for non-contact delivery of the testing results to a nearby electronic computer device.

2. The device of claim 1, wherein the communication board is extractable and recyclable and includes near field communication technology for wireless transfer of collected data and results output of the microRNA analysis conducted on the sensor holder board.

3. The device of claim 2, wherein the communication module is configured to send encrypted patient data capable of being analyzed and uploaded on an analyzing device for post-processing.

4. A method for the detection of cancer conducted by a patient comprising of:
a. scanning a QR code printed in the box of the urine liquid container;
b. downloading and installing an application on an electronic computer device;
c. conducting a calibration test to set the no-load response;
d. collecting urine in the liquid vessel;
e. turning the liquid vessel upside down to allow the liquid content to touch the sensor holder board containing the GFET arrays;
f. activating an NFC connection approaching an electronic computer device to the smart lid;
g. waiting until an acoustic signal warns the test is completed and;
h. sending the result to a physician for analysis of the results.
